(19) United States
(12) Patent Application Publication
(10) Pub. No.: US 2017/0238883 A1
(43) Pub. Date: Aug. 24, 2017

(54) SIGNAL PROCESSING SYSTEM AND METHOD FOR MEDICAL IMAGING SYSTEM USING MULTI THRESHOLD VOLTAGE

(71) Applicant: SOGANG UNIVERSITY RESEARCH FOUNDATION, Seoul (KR)

(72) Inventors: Yong CHOI, Seoul (KR); Kyu Bom KIM, Goyang-si, Gyeonggi-do (KR)

(73) Assignee: SOGANG UNIVERSITY RESEARCH FOUNDATION, Seoul (KR)

(21) Appl. No.: 15/523,349

(22) PCT Filed: Sep. 23, 2015
(86) PCT No.: PCT/KR2015/009994

§ 371 Date: Apr. 28, 2017

(30) Foreign Application Priority Data
Oct. 30, 2014 (KR) 10-2014-0149409

(51) Int. Cl. A61B 6/03; G01T 1/161; G06T 7/00

(52) U.S. Cl. CPC A61B 6/037; G06T 7/0012; G01T 1/161; G06T 2207/10108; G06T 2207/10116; G06T 2207/10104

(57) ABSTRACT

Disclosed herein is a signal processing system for medical imaging system using a multi threshold voltage. The signal processing system for medical imaging system may include: a signal detector detecting radiation emitted from radiopharmaceutical products injected into an object or radiation irradiated to the object and transmitting the object to generate and output a radiation detection signal; an analog signal processor receiving radiation detection signals for each channel output from the signal detector and a plurality of preset different threshold voltages and each comparing the received radiation detection signals with signals depending on the plurality of different threshold voltages to generate and output a plurality of trigger signals; and a digital signal processor receiving the trigger signals and acquiring energy information, time information, and position information representing detailed information on the radiation detection within the object on the basis of the received trigger signals.

### CROSS-REFERENCE TO RELATED APPLICATION

This application is based on and claims the benefit of priority to Korean Patent Application No. 10-2014-0149409 filed in the Korean Intellectual Property Office on Oct. 30, 2014, the disclosure of which is incorporated herein in its entirety by reference.

### TECHNICAL FIELD

The present disclosure relates to a signal processing system and method for medical imaging system using a multi threshold voltage, and more particularly, to a signal processing system and method for medical imaging system using a multi threshold voltage capable of easily figuring out energy information, time information and position information of emitted radiation using a radiation detection signal of a medical image field and a plurality of threshold voltages having different values.

### BACKGROUND

Recently, with the development of IT technologies, medical imaging systems that non-invasively display an inside of a living body in an image form to provide information required for accurate diagnosis of a disease have been widely used in a medical field. Among these medical imaging systems, as tomography image acquisition equipment, there are X-ray computed tomography (CT), magnetic resonance imaging (MRI), nuclear medicine imaging equipment, or the like. In particular, the X-ray computed tomography (CT) and the magnetic resonance imaging (MRI) provide a detailed anatomical image of a human body and the nuclear medicine imaging equipment using radioisotope provides an image displaying a physiological phenomenon in a human body.

There is a problem in that development cost of a data signal processing system used for the medical imaging system to acquire a medical image is increased upon multi-channel extension and a size of the data signal processing system is also increased. In addition, to reduce the size of the system upon the multi-channel extension within the signal processing system, ASIC is used, which results in an increase in costs.

### SUMMARY

The present disclosure has been made to solve the above-mentioned problems occurring in the prior art while advantages achieved by the prior art are maintained intact.

An aspect of the present disclosure provides a signal processing system and method for medical imaging system capable of easily figuring out information on energy, time, location, or the like of radiation by applying a plurality of threshold voltages having different values to a comparator and comparing radiation detection signals with the plurality of threshold voltages to generate trigger signals, not using an analog-to-digital converter (ADC) or a time-to-digital converter (TDC).

According to an exemplary embodiment of the present disclosure, a signal processing system for medical imaging system using a multi threshold voltage includes: a signal detector detecting radiation emitted from radiopharmaceutical products injected into an object or radiation irradiated to the object and transmitting the object to generate and output a radiation detection signal; an analog signal processor receiving radiation detection signals for each channel output from the signal detector and a plurality of preset different threshold voltages and each comparing the received radiation detection signals with signals depending on the plurality of different threshold voltages to generate and output a plurality of trigger signals; and a digital signal processor receiving the trigger signals and acquiring energy information, time information, and position information representing detailed information on the radiation detection within the object on the basis of the trigger signals.

The signal detector may include one of a single photon emission computed tomography (SPECT) detector, a gamma camera, and an X-ray detector, a positron emission tomography detector.

The analog signal processor may include: an amplifier receiving radiation detection signals for each channel output from the signal detector to amplify the radiation detection signals so as to make a gain of the radiation detection signals uniform; and a plurality of comparators receiving the amplified radiation detection signals and a plurality of preset threshold voltage values having different values to compare the radiation detection signals and signals depending on the plurality of threshold voltage values with each other to generate and output a plurality of trigger signals.

The analog signal processor may further include an exclusive OR operator receiving each of the plurality of trigger signals output from the plurality of comparators and performing an exclusive OR operation between the plurality of received trigger signals to output an operation result.

The digital signal processor may receive the plurality of output trigger signals from the analog signal processor, stores a counter value output from a counter being driven therein in response to the plurality of received trigger signals, and then operates detected gamma-ray energy information and arrival time information of radiation on the basis of the stored counter value.

The digital signal processor may include: a first information acquisition module integrating a width of the counter value to acquire detected gamma-ray energy information; and a second information acquisition module acquiring an output time of the counter value output from the counter as arrival time information.

The digital signal processor may be configured of a field programmable gate array (FPGA).

The digital signal processor may further include a third information acquisition module acquiring reaction position information of radiation on the basis of a location of an input/output (I/O) pin of the FPGA receiving the comparison signal.

According to another exemplary embodiment of the present disclosure, a signal processing method for medical imaging system using a multi threshold voltage includes: detecting, by a signal detector, radiation emitted from radiopharmaceutical products injected into an object or radiation irradiated to the object and transmitting the object to generate and output a radiation detection signal; receiving, by an analog signal processor, radiation detection signals for each channel output from the signal detector to amplify the radiation detection signals so as to make a gain of the radiation detection signals uniform; receiving, the analog signal processor, a plurality of preset different threshold voltage values and comparing the amplified radiation detection signals and signals depending on the plurality of different threshold voltage values with each other to generate and output trigger signals; and receiving, by a digital signal processor, the trigger signals output from the analog signal processor and acquiring energy information, time information, and position information representing detailed information on the radiation detection based on the received trigger signals.

The comparing, by the analog signal processor, the radiation detection signals with the signals depending on the plurality of threshold voltage values to generate the trigger signals may include: receiving, by an amplifier, radiation detection signals for each channel output from the signal detector to amplify the radiation detection signals so as to make a gain of the radiation detection signals uniform; and receiving, by a plurality of comparators, the plurality of amplified radiation detection signals and the plurality of preset threshold voltage values to compare the amplified radiation detection signals and the signals depending on the plurality of received threshold voltage values with each other to output a plurality of trigger signals.

The comparing, by the analog signal processor, the radiation detection signals with the signals depending on the plurality of threshold voltage values to generate the trigger signals may further include receiving, by an exclusive OR operator, a plurality of trigger signals output of the plurality of comparators, respectively, performing an exclusive OR operation between the plurality of received trigger signals, and outputting the operation result.

In the acquiring, by the digital signal processor, the energy information, the time information, and the position information on the radiation detection, the plurality of trigger signals output from the analog signal processor may be received, a counter value output from a counter being driven therein in response to the plurality of received trigger signals may be stored, and then detected gamma-ray energy information and arrival time information on the radiation detection within the object may be operated on the basis of the stored counter value.

The acquiring, by the digital signal processor, the energy information, the time information, and the position information on the radiation detection may include: integrating a width of the counter value to acquire detected gamma-ray energy information; and acquiring an output time of the counter value output from a counter being driven therein as arrival time information.

The acquiring, by the digital signal processor, the energy information, the time information, and the position information on the radiation detection may further include: acquiring reaction position information of radiation on the basis of a location of an input/output (I/O) pin of FPGA receiving the comparison signal.

### BRIEF DESCRIPTION OF THE DRAWINGS

The above and other objects, features and advantages of the present disclosure will be more apparent from the following detailed description taken in conjunction with the accompanying drawings:

FIG. 1 is a conceptual diagram of a signal processing system for medical imaging system using a multi threshold voltage according to an exemplary embodiment of the present disclosure;

FIG. 2 is a flow chart of a signal processing method for a medical imaging system using a multi threshold voltage according to another exemplary embodiment of the present disclosure;

FIG. 3 is a diagram illustrating an input/output signal of a comparator;

FIG. 4 is a timing diagram illustrating a plurality of trigger signals responding to an internal clock;

FIG. 5 is a diagram illustrating pin setting of FPGA;

FIG. 6 is a conceptual diagram of a signal processing system for medical imaging system using a multi threshold voltage according to another exemplary embodiment of the present disclosure; and

FIG. 7 is a timing diagram illustrating an exclusive OR operation between trigger signals within an analog signal processor.

### DETAILED DESCRIPTION

Terms and words used in the present specification and claims are not to be construed as a general or dictionary meaning but are to be construed as meaning and concepts meeting the technical ideas of the present disclosure based on a principle that the inventors can appropriately define the concepts of terms in order to describe their own disclosures in best mode. Therefore, configurations described in exemplary embodiments and the accompanying drawings of the present disclosure do not represent all of the technical spirits of the present disclosure, but are merely most preferable embodiments. Therefore, the present disclosure should be construed as including all the changes, equivalents, and substitutions included in the spirit and scope of the present disclosure at the time of filing this application.

Sizes of each component illustrated in the drawings or specific parts forming the components may be exaggerated or simplified for clarity and convenience. Therefore, the size of each component does not exactly reflect its real size. Further, when it is determined that the detailed description of the known function or configuration related to the present disclosure may obscure the gist of the present disclosure, the detailed description thereof will be omitted.

A signal processing system for medical imaging system using a multi threshold voltage according to an exemplary embodiment of the present disclosure may include: a signal detector detecting radiation emitted from radiopharmaceutical products injected into an object or radiation irradiated to the object and transmitting the object to generate and output a radiation detection signal; an analog signal processor receiving radiation detection signals for each channel output from the signal detector and a plurality of preset different threshold voltages and each comparing the received radiation detection signals with signals depending on the plurality of different threshold voltages to generate and output a plurality of trigger signals; and a digital signal processor receiving the trigger signals and acquiring energy information, time information, and position information representing detailed information on the radiation detection within the object on the basis of the received trigger signals.

A signal processing method for medical imaging system using a multi threshold voltage according to an exemplary embodiment of the present disclosure may include: detecting, by a signal detector, radiation emitted from radiopharmaceutical products injected into an object or radiation irradiated to the object and transmitting the object to generate and output a radiation detection signal; receiving, by an analog signal processor, radiation detection signals for each channel output from the signal detector to amplify the radiation detection signals so as to make a gain of the radiation detection signals uniform; and receiving, the analog signal processor, a plurality of preset different threshold voltage values and comparing the amplified radiation detection signals and signals depending on the plurality of different threshold voltage values with each other to generate and output trigger signals; and receiving, by a digital signal processor, the trigger signals output from the analog signal processor and acquiring energy information, time information, and position information representing detailed information on the radiation detection based on the received trigger signals.

Hereinafter, the present disclosure will be described in detail with reference to exemplary embodiments of the present disclosure and the accompanying drawings so that a person having ordinary skill in the art to which the present disclosure pertains may easily perform the present disclosure. However, the present disclosure may be implemented in various forms and therefore is not limited to the embodiments described herein.

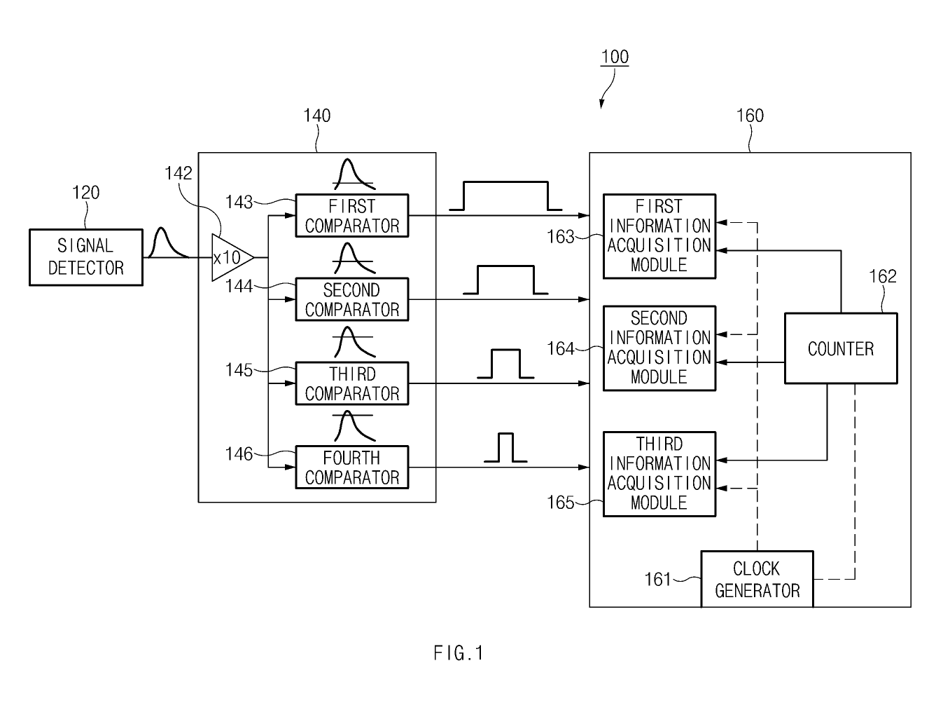

Hereinafter, a signal processing system for medical imaging system using a multi threshold voltage according to an exemplary embodiment of the present disclosure will be described in detail with reference to FIG. 1.

FIG. 1 is a conceptual diagram of a signal processing system for medical imaging system using a multi threshold voltage according to an exemplary embodiment of the present disclosure.

As illustrated in FIG. 1, a signal processing system 100 for medical imaging system using a multi threshold voltage according to an exemplary embodiment of the present disclosure includes a signal detector 120, an analog signal processor 140, and a digital signal processor 160.

The signal detector 120 detects radiation emitted from radiopharmaceutical products injected into an object or radiation irradiated to the object and transmitting the object to generate and output a radiation detection signal representing in vivo distribution or distribution in internal organs. The signal detector may include one of a single photon emission computed tomography (SPECT) detector, a gamma camera, and an X-ray detector, a positron emission tomography detector and the radiation detection signal output may be output in a Gaussian signal or semi-Gaussian signal form.

In this case, the SPECT detector is a single photon emission computed tomography detector and may inject radioisotope emitting a single photon in an object or a human body and then perform tomography imaging on a distribution thereof to assess a biochemical change or a functional problem of an object.

Further, the gamma camera is a camera that detects a radioactive tracer injected into an object or a human body to record a shape of internal organs in the object or the human body and may confirm a specific distribution of the corresponding internal organ when an organotropy material representing the radioisotope is given to a patient.

The PET detector is positron emission tomography and injects the radiopharmaceutical products emitting a positron within the object or the human body by intravenous injection or suction and then detects two gamma rays having energy of 0.511 MeV emitted in a 180° direction by an annihilation reaction after positron emission from positron emission radioisotope within the object or the human body, thereby configuring an image.

The analog signal processor 140 receives radiation detection signals for each channel output from the signal detector 120 and each of the plurality of preset different threshold voltages from the outside and compares the received radiation detection signals with signals depending on the plurality of different threshold voltages, respectively, to generate and output a plurality of trigger signals. The analog signal processor 140 includes an amplifier 142, a signal synthesizer (not illustrated), and a plurality of comparators 143, 144, 145, and 146.

The amplifier 142 receives the radiation detection signals for each channel output from the signal detector 120 to amplify the radiation detection signals so as to make a gain of the radiation detection signals uniform.

The plurality of comparators 143, 144, 145, and 146 receive the synthesized radiation detection signals and the plurality of preset threshold voltage values having different values to compare the radiation detection signals and the signals depending on the plurality of threshold voltage values with each other to generate and each output the plurality of trigger signals.

The digital signal processor 160 receives each of the plurality of trigger signals from the comparators 143, 144, 145, and 146 within the analog signal processor 140 and acquires energy information, arrival time information, and reaction position information that are detected from the object in response to the received trigger signals or the detailed information on the radiation transmitted from the object. In particular, the digital signal processor 160 may be configured of a field programmable gate array (FPGA) and receives the plurality of trigger signals output from the analog signal processor 140, stores a counter value of a counter 162 that is being operated therein in response to the plurality of received trigger signals, and then uses the stored counter value to operate the detected gamma-ray energy information and the arrival time information of the radiation.

The digital signal processor 160 includes a first information acquisition module 163, a second information acquisition module 164, and a third information acquisition module 165.

The first information acquisition module 163 integrates a width of the stored counter value to acquire the detected gamma-ray energy information.

The second information acquisition module 164 acquires an output time of the counter value output from the counter 162 as the arrival time information.

The third information acquisition module 165 uses locations of input/output (I/O) pins of the FPGA receiving the trigger signals from the plurality of comparators 143, 144, 145, and 146 to acquire the reaction position information of the radiation.

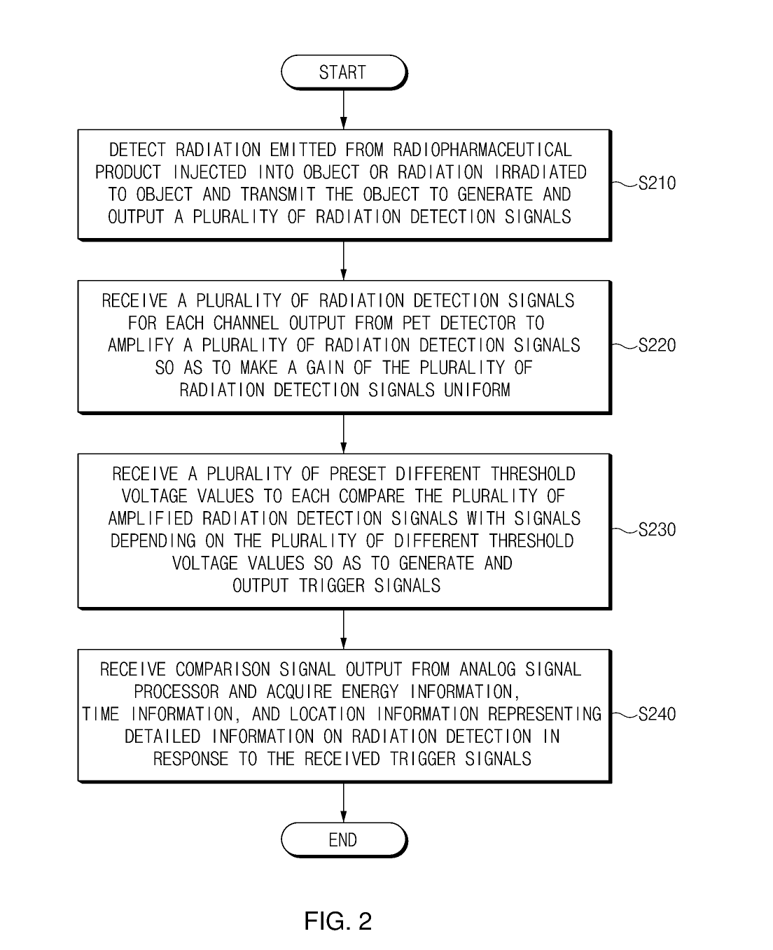

Hereinafter, a signal processing method for medical imaging system using a multi threshold voltage will be described in detail with reference to FIG. 2.

FIG. 2 is a flow chart of a signal processing method for medical imaging system using a multi threshold voltage according to another exemplary embodiment of the present disclosure.

As illustrated in FIG. 2, the signal processing method for medical imaging system using a multi threshold voltage according to another exemplary embodiment of the present disclosure detects, by the signal detector 120, the radiation emitted from the radiopharmaceutical products injected into the object or the radiation irradiated to the object and transmitting the object to generate and output the radiation detection signal representing in vivo distribution or distribution in internal organs (S210).

The analog signal processor 140 receives the radiation detection signals for each channel output from the signal detector 120 to amplify the radiation detection signals so as to make the gain of the radiation detection signal uniform (S220).

The analog signal processor 140 receives the plurality of preset different threshold voltage values to compare the amplified radiation detection signals and the signals depending on the plurality of different threshold voltage values with each other to generate and output the trigger signals (S230).

Describing in more detail the process of generating the trigger signals, the amplifier 142 receives the radiation detection signals for each channel output from the signal detector 120 to amplify the radiation detection signals so as to make the gain of the radiation detection signals uniform.

Next, the plurality of comparators 143, 144, 145, and 146 receive the amplified radiation detection signals and the plurality of preset threshold voltage values having different values to compare the amplified radiation detection signals with the signals depending on the plurality of received threshold voltage values, respectively, thereby outputting the plurality of trigger signals, respectively.

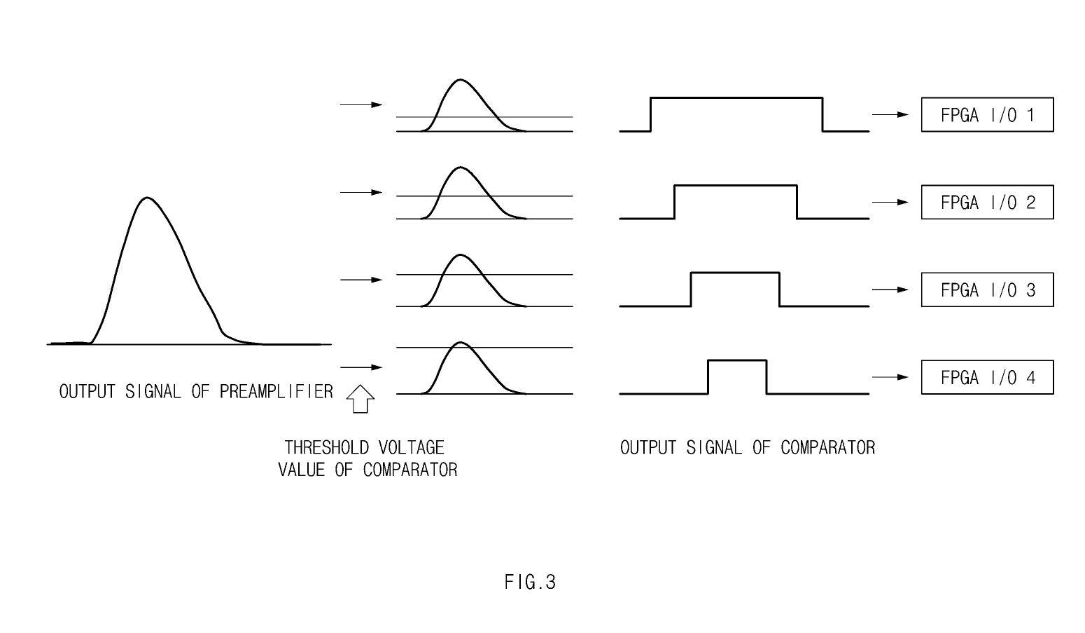

FIG. 3 is a diagram illustrating an input/output signal of a comparator.

As illustrated in FIG. 3, an optical sensor output signal received from the signal detector 120 is amplified by the amplifier 142 that is located within the analog signal processor 140. The amplified signal passes through each of the plurality of comparators 143, 144, 145, and 146 having different threshold voltages within the analog signal processor 140 to be converted into the comparison signal and the converted comparison signal is input to the digital signal processor 160.

According to the exemplary embodiment of the present disclosure, four comparators 143, 144, 145, and 146 are used to output four different trigger signals. At this point, the number of threshold voltages may be changed depending on the number of comparators set by a user.

The digital signal processor 160 receives each of the plurality of trigger signals output from the analog signal processor 140 through the plurality of input/output ports (I/O pins) and acquires the energy information, the time information, and the position information representing the detailed information on the radiation detection on the basis of the received comparison signal (S240). The digital signal processor 160 receives the plurality of trigger signals output from the analog signal processor 140, stores the counter value of the counter that is being operated therein on the basis of the received comparison signal, and then operates the detected gamma-ray energy information and the arrival time information on the radiation detected from the object on the basis of the counter value.

At this point, the digital signal processor 160 may be configured of the FPGA, in particular, the FPGA is operated by an internal clock smaller than 350 MHz generated from a clock generator 161 therein and when the plurality of trigger signals output from each of the comparators 143, 144, 145, and 146 are input to the digital signal processor 160, the counter value of the counter 162 that is operated by the internal clock is separately stored.

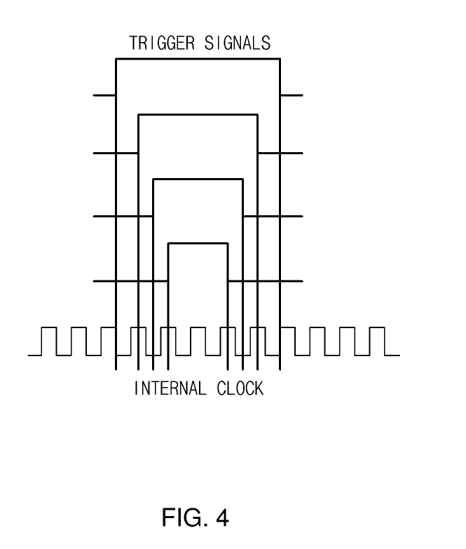

FIG. 4 is a timing diagram illustrating a comparison signal responding to an internal clock.

As illustrated in FIG. 4, it can be appreciated that when the comparison signal output from the first comparator 143 in which the lowest threshold voltage is set is input to the digital signal processor 160, the highest counter value is stored, and to the contrary, when the comparison signal output from the fourth comparator 146 in which the highest threshold voltage is set is input to the digital signal processor 160, the lowest counter value is stored.

As such, different trigger signals for each comparator are output, different counter values are stored depending on each of the output trigger signals, and the detected gamma-ray energy information and the arrival time information on the radiation detected from the object using the stored counter value are operated.

Hereinafter, the operation process of the detected gamma-ray energy information and the arrival time information on the radiation detected from the object will be described below in detail.

First, the detected gamma-ray energy information of the radiation may be operated by integrating the width of the stored counter value.

Further, the arrival time information may be operated on the basis of a course time stamp, a time work correction value, and an energy quantity of the radiation injected into the object.

In addition, the reaction position information of the radiation is acquired on the basis of the locations of the input/output (I/O) pins of the FPGA that receives the trigger signals, respectively.

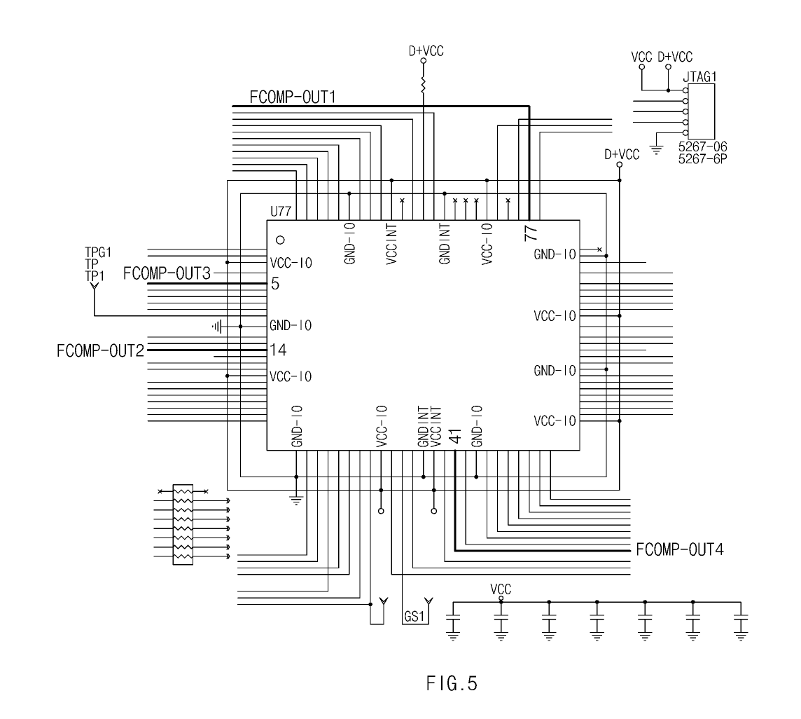

FIG. 5 is a diagram illustrating an example of the pin setting of the FPGA.

As illustrated in FIG. 5, the first to fourth trigger signals are output from the four comparators 143, 144, 145, and 146 located within the analog signal processor 140. The output first comparison signal is input to a first input/output port FCOMP-OUT1 of the digital signal processor 160, the second comparison signal is input to a second input/output port FCOMP-OUT2 of the digital signal processor 160, the third comparison signal is input to a third input/output port FCOMP-OUT3 of the digital signal processor 160, and the fourth comparison signal is input to a fourth input/output port FCOM-OUT4 of the digital signal processor 160. At this point, the input/output port of the digital signal processor 160 receiving the first to fourth trigger signals may confirm the reaction locations of the radiation of the first to fourth trigger signals input through the locations of the preset pins.

Further, in the foregoing analog signal processing process, the used number of output channels may be reduced by an exclusive OR operator.

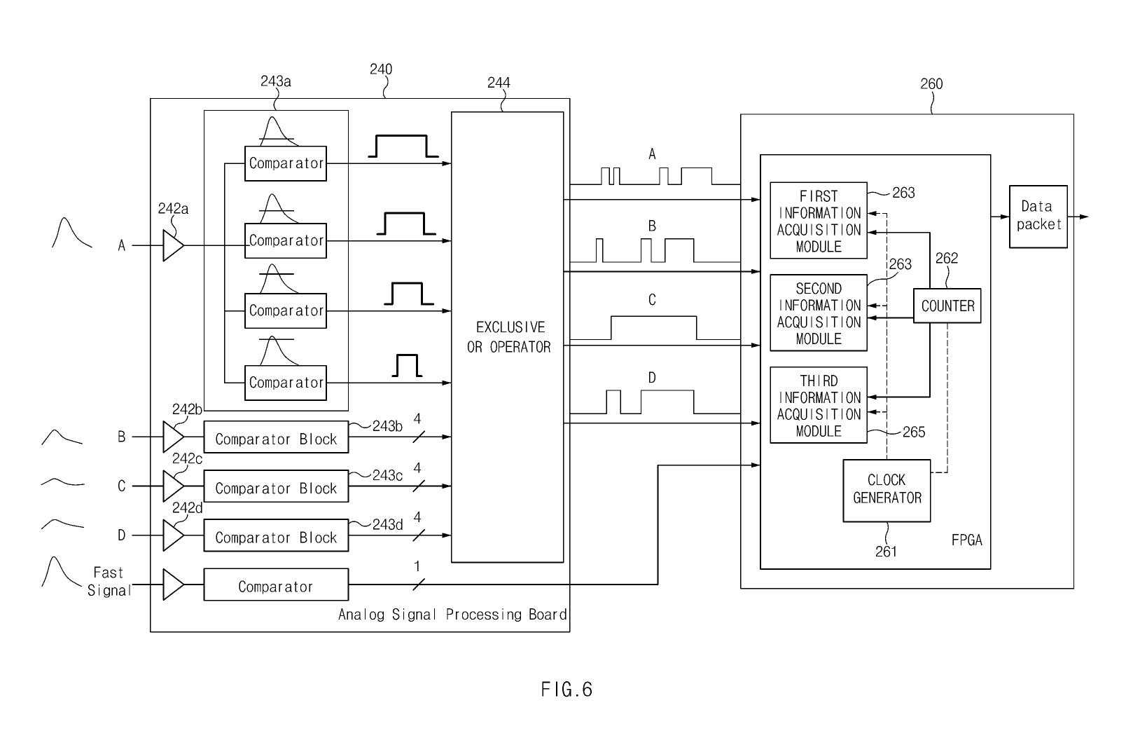

FIG. 6 is a conceptual diagram of a signal processing system for medical imaging system using a multi threshold voltage according to another exemplary embodiment of the present disclosure.

The signal processing system for medical imaging system using a multi threshold voltage according to the exemplary embodiment of the present disclosure illustrated in FIG. 6 is very similar to the system configuration described with reference to FIG. 1 and the configuration having a difference from that of the foregoing system will be mainly described.

As illustrated in FIG. 6, a signal processing system 200 for medical imaging system using a multi threshold voltage according to another exemplary embodiment of the present disclosure includes a signal detector (not illustrated), an analog signal processor 240, and a digital signal processor 260. Here, the configuration of the signal detector and the digital signal processor 260 is the same as that already described with reference to FIG. 1, and therefore the detailed description thereof will be omitted.

The analog signal processor 240 includes a plurality of amplifiers 242a, 242b, 242c, and 242d, a plurality of comparator blocks 243a, 243b, 243c, and 243d including a plurality of comparators, and an exclusive OR operator 244.

The plurality of amplifiers 242a, 242b, 242c, and 242d receives the radiation detection signals for each channel detected by the signal detector and amplifies the received radiation detection signals to make the gain of the radiation detection signals uniform.

The amplification signals received by the plurality of first to fourth comparators included in one comparator block and the signals depending on the plurality of received threshold voltage values set to have different values are compared with each other to generate and output a plurality of trigger signals, respectively.

The exclusive OR operator 244 receives the plurality of trigger signals output from the first to fourth comparators, respectively, within one comparator block and each performs the exclusive OR operation between the plurality of received trigger signals to output XOR output signals and transfer the output XOR output signals to the digital signal processor 260.

At this point, the exclusive OR operator 244 performs the exclusive OR operation between the signals output from each comparator block. That is, as illustrated in FIG. 6, four comparators having a total of four different threshold voltage values each compare one amplification signal, and therefore four comparators included within one comparator block output four trigger signals and the exclusive OR operator performs the exclusive OR operation on the output four trigger signals to generate and output a total of one XOR output signal. Consequently, the exclusive OR operator 244 generates and outputs one XOR output signal per the comparator block.

Therefore, if it is assumed that a total of four comparator blocks each include four comparators, a total of 16 output signals are generated, but when the exclusive OR operator is used, only one output signal per the comparator block is generated to generate a total of four output signals, such that the number of output channels used in the medical imaging system may be greatly reduced to ¼.

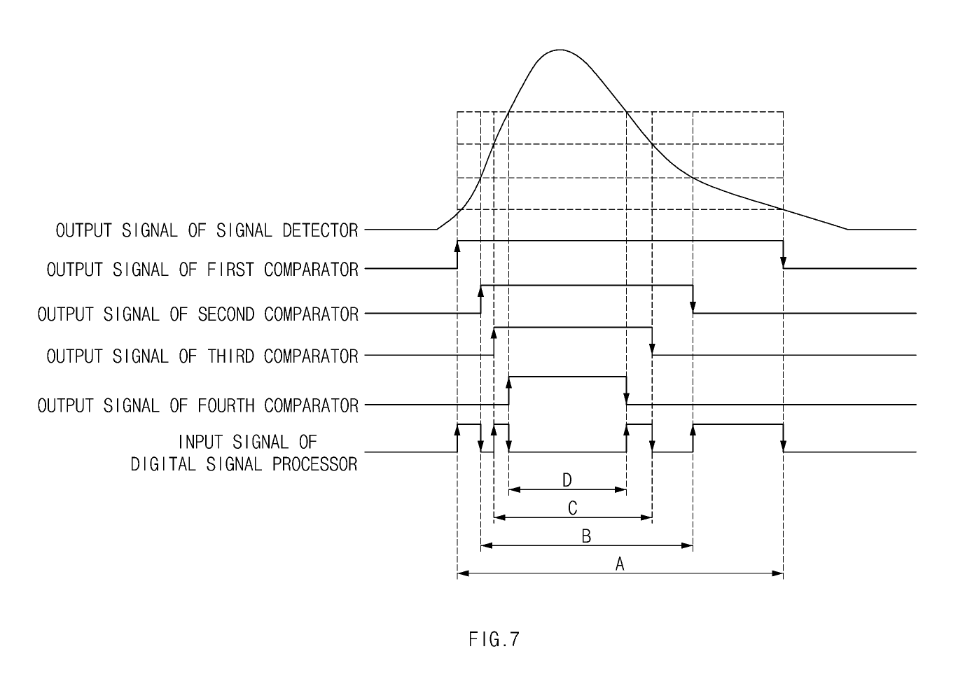

The signals processed within the analog signal processor may confirm the relationship between the signals on the basis of the timing diagram illustrated in FIG. 7.

Next, the digital signal processor 260 receives the XOR output signal output through the exclusive OR operator and determines the time to first receive the XOR output signal as the time to detect the radiation within the object.

Further, the digital signal processor 260 may measure a signal from a first high state of the XOR output signal to a second high state of the XOR output signal and then analyze the signal to measure the radiation detection energy quantity within the object.

As such, the number of output channels actually used in the medical imaging system is greatly reduced by the exclusive OR operator 244 included in the analog signal processor 240, thereby greatly shortening the processing time for the data acquisition.

Further, the signal processing system and method for medical imaging system using a multi threshold voltage may be stored in a computer-readable recording medium recorded with a program executed by a computer. At this point, the computer readable recording medium includes all kinds of recording apparatuses in which data readably by a computer system are stored. An example of the computer readable recording apparatus may include ROM, RAM, CD-ROM, DVD±ROM, DVD-RAM, a magnetic tape, a floppy disk, a hard disk, an optical data storage apparatus, or the like. Further, the computer readable recording medium is distributed in the computer apparatus connected by a network and may be stored with a computer readable code in a distributed manner and executed.

According to the exemplary embodiments of the present disclosure, the signal processing system and method for medical imaging system using a multi threshold voltage may apply the plurality of threshold voltages having different values to the comparator, separate the signals output from the signal detector based on the plurality of applied threshold voltages, and then easily figure out the information of energy, time, location, or the like for the radiation detection based on the separated signal.

Further, according to the exemplary embodiments of the present disclosure, the signal processing system and method for medical imaging system using a multi threshold voltage may separate the signals output from the signal detector based on the plurality of different threshold voltages applied to the comparator to prevent the size and costs of the signal processing system from increasing compared to the related art figuring out the information of energy, time, location, or the like for the radiation detection using the ADC or the TDC.

In addition, according to the exemplary embodiments of the present disclosure, the signal processing system and method for medical imaging system using a multi threshold voltage may perform the exclusive OR operation between the plurality of trigger signals output from the comparator within the analog signal processor to greatly reduce the number of output channels used in the medical imaging system, thereby greatly shortening the processing time for data acquisition.

According to the exemplary embodiments of the present disclosure, the signal processing system and method for medical imaging system using a multi threshold voltage may apply the plurality of threshold voltages having different values to the comparator, separate the signals output from the signal detector based on the plurality of applied threshold voltages, and then easily figure out the information of energy, time, location, or the like for the radiation detection based on the separated signal.

Further, according to the exemplary embodiments of the present disclosure, the signal processing system and method for medical imaging system using a multi threshold voltage may separate the signals output from the signal detector based on the plurality of different threshold voltages applied to the comparator to prevent the size and costs of the signal processing system from increasing, compared to the related art figuring out the information of energy, time, location, or the like for the radiation detection using the ADC or the TDC.

In addition, according to the exemplary embodiments of the present disclosure, the signal processing system and method for medical imaging system using a multi threshold voltage may perform the exclusive OR operation between the plurality of trigger signals output from the comparator within the analog signal processor to greatly reduce the number of output channels used in the medical imaging system, thereby greatly shortening the processing time for data acquisition.

Hereinabove, although the present disclosure has been described with reference to exemplary embodiments and the accompanying drawings, the present disclosure is not limited thereto, but may be variously modified and altered by those skilled in the art to which the present disclosure pertains without departing from the spirit and scope of the present disclosure claimed in the following claims.

1. A signal processing system for medical imaging system using a multi threshold voltage, comprising:
a signal detector detecting radiation emitted from radiopharmaceutical products injected into an object or radiation irradiated to the object and transmitting the object to generate and output a radiation detection signal;
an analog signal processor receiving radiation detection signals for each channel output from the signal detector and a plurality of preset different threshold voltages and each comparing the received radiation detection signals with signals depending on the plurality of different threshold voltages to generate and output a plurality of trigger signals; and
a digital signal processor receiving the trigger signals and acquiring energy information, time information, and position information representing detailed information on the radiation detection within the object on the basis of the received trigger signals.

2. The signal processing system according to claim 1, wherein the signal detector includes one of a single photon emission computed tomography (SPECT) detector, a gamma camera, and an X-ray detector, a positron emission tomography detector.

3. The signal processing system according to claim 1, wherein the analog signal processor includes:
an amplifier receiving radiation detection signals for each channel output from the signal detector to amplify the radiation detection signals so as to make a gain of the radiation detection signals uniform; and
a plurality of comparators receiving the amplified radiation detection signals and a plurality of preset threshold voltage values having different values to compare the radiation detection signals and signals depending on the plurality of threshold voltage values with each other to generate and output a plurality of trigger signals.

4. The signal processing system according to claim 3, wherein the analog signal processor further includes an exclusive OR operator receiving each of the plurality of trigger signals output from the plurality of comparators and performing an exclusive OR operation between the plurality of received trigger signals to output an operation result.

5. The signal processing system according to claim 1, wherein the digital signal processor receives a plurality of output signals from the analog signal processor, stores a counter value output from a counter being driven therein in response to the plurality of received output signals, and then operates detected gamma-ray energy information and arrival time information of radiation on the basis of the stored counter value.

6. The signal processing system according to claim 5, wherein the digital signal processor includes:
a first information acquisition module integrating a width of the counter value to acquire detected gamma-ray energy information; and
a second information acquisition module acquiring an output time of the counter value output from the counter as arrival time information.

7. The signal processing system according to claim 5, wherein the digital signal processor is configured of a field programmable gate array (FPGA).

8. The signal processing system according to claim 7, wherein the digital signal processor further includes a third information acquisition module acquiring reaction position information of radiation on the basis of a location of an input/output (I/O) pin of the FPGA receiving the comparison signal.

9. A signal processing method for medical imaging system using a multi threshold voltage, comprising:
detecting, by a signal detector, radiation emitted from radiopharmaceutical products injected into an object or radiation irradiated to the object and transmitting the object to generate and output a radiation detection signal;
receiving, by an analog signal processor, radiation detection signals for each channel output from the signal detector to amplify the radiation detection signals so as to make a gain of the radiation detection signals uniform;
receiving, the analog signal processor, a plurality of preset different threshold voltage values and comparing the amplified radiation detection signals and signals depending on the plurality of different threshold voltage values with each other to generate and output trigger signals; and
receiving, by a digital signal processor, the trigger signals output from the analog signal processor and acquiring energy information, time information, and position information representing detailed information on the radiation detection based on the received trigger signals.

10. The signal processing method according to claim 9, wherein the comparing, by the analog signal processor, the radiation detection signals with the signals depending on the plurality of threshold voltage values to generate the trigger signals includes:
receiving, by an amplifier, radiation detection signals for each channel output from the signal detector to amplify the radiation detection signals so as to make a gain of the radiation detection signals uniform; and
receiving, by a plurality of comparators, the plurality of amplified radiation detection signals and the plurality of preset threshold voltage values to compare the amplified radiation detection signals and the signals depending on the plurality of received threshold voltage values with each other to output a plurality of trigger signals.

11. The signal processing method according to claim 10, wherein the comparing, by the analog signal processor, the radiation detection signals with the signals depending on the plurality of threshold voltage values to generate the trigger signals further includes receiving, by an exclusive OR operator, a plurality of trigger signals output of the plurality of comparators, respectively, performing an exclusive OR operation between the plurality of received trigger signals, and outputting the operation result.

12. The signal processing method according to claim 9, wherein in the acquiring, by the digital signal processor, the energy information, the time information, and the position information on the radiation detection, the plurality of trigger signals output from the analog signal processor are received, a counter value output from a counter being driven therein in response to the plurality of received trigger signals is stored, and then detected gamma-ray energy information and arrival time information on the radiation detection within the object are operated on the basis of the stored counter value.

13. The signal processing method according to claim 12, wherein the acquiring, by the digital signal processor, the energy information, the time information, and the position information on the radiation detection includes:
integrating a width of the counter value to acquire detected gamma-ray energy information; and
acquiring an output time of the counter value output from the counter being driven therein as arrival time information.

14. The signal processing method according to claim 13, wherein the acquiring, by the digital signal processor, the energy information, the time information, and the position information on the radiation detection further includes acquiring reaction position information of radiation on the basis of a location of an input/output (I/O) pin of FPGA receiving the comparison signal.

15. (canceled)
